(19) United States
(12) Patent Application Publication
(10) Pub. No.: US 2021/0353133 A1
(43) Pub. Date: Nov. 18, 2021

(54) ENDOSCOPE

(71) Applicant: OLYMPUS CORPORATION, Tokyo (JP)

(72) Inventor: Hidetoshi SAITO, Hanno-shi (JP)

(73) Assignee: OLYMPUS CORPORATION, Tokyo (JP)

(21) Appl. No.: 17/391,766

(22) Filed: Aug. 2, 2021

Related U.S. Application Data
(63) Continuation of application No. PCT/JP2019/004446, filed on Feb. 7, 2019.

(51) Int. Cl. A61B 1/005

(52) U.S. Cl. CPC A61B 1/0052; A61B 1/0057

(57) ABSTRACT

An endoscope includes: an insertion portion including a bending portion; a lever configured to be capable of, by being tilted, pulling a bending operation wire that bends the bending portion; and a bending operation mechanism including a play part configured to, when the lever is tilted in a predetermined direction, maintain the bending portion in a state of not bending, up to a predetermined tilting amount. The bending operation mechanism includes a sub-rotation pivot provided at a position that is away in an axial direction of the lever from tilting supports of the lever in the respective tilting directions and configured to tilt the lever in a predetermined direction, and a stopper configured to, when the lever is rotated by a predetermined angle via rotation of the sub-rotation pivot, stop the rotation of the sub-rotation pivot through abutment with the lever.

### CROSS REFERENCE TO RELATED APPLICATION

This application is a continuation application of PCT/JP2019/004446 filed on Feb. 7, 2019, the entire contents of which are incorporated herein by this reference.

### BACKGROUND OF THE INVENTION

### 1. Field of the Invention

The present invention relates to an endoscope including a lever capable of, by being tilted, selectively and actively bending a bending portion provided in an insertion portion in any one of upward, downward, leftward and rightward directions or a composite direction of two directions of the upward, downward, leftward and rightward directions.

### 2. Description of the Related Art

In recent years, endoscopes have widely been used in the medical field. An endoscope used in the medical field enables observing a site to be examined inside a body cavity, which is a subject, by an elongated insertion portion being inserted in the body cavity.

A configuration in which a bending portion that is actively and selectively bendable in any one of upward, downward, leftward and rightward directions or a composite direction of two directions of the upward, downward, leftward and rightward directions is provided on the distal end side of an insertion portion of an endoscope is publicly known.

By being bent, the bending portion enhances capability of advancement of the insertion portion at a flexed part inside a subject and also changes an observation direction of an observation optical system provided in a distal end portion located on the distal end side in a longitudinal axis direction of the insertion portion relative to the bending portion in the insertion portion.

As an example, two pairs of, that is, four bending operation wires with respective distal ends in a longitudinal axis direction fixed to the bending portion are inserted inside an insertion portion of an endoscope and inside an operation portion of the endoscope, the operation portion being provided in such a manner as to be continuous with a proximal end in the longitudinal axis direction of the insertion portion.

Any one of the four bending operation wires or any two of the four bending operation wires are pulled by a bending operation device provided in the operation portion of the endoscope.

Accordingly, the bending portion is actively and selectively bendable in any one of four directions of upward, downward, leftward and rightward directions or a composite direction of two directions of the upward, downward, leftward and rightward directions.

A configuration of the bending operation device configured to pull the bending operation wires is publicly known. For example, International Publication No. WO2018/029916 discloses a configuration of an endoscope using a known joystick device for the bending operation device.

The joystick device actively and selectively bends a bending portion by bending operation wires being pulled by a bending operation mechanism along with an operation to tilt a lever.

More specifically, in the endoscope disclosed in International Publication No. WO2018/029916, a lever of the joystick device is configured to be selectively tiltable in a first direction corresponding to an upward direction in bending directions of the bending portion or a second direction corresponding to a downward direction in the bending directions via a first rotation pivot provided at a tilting support. Consequently, the bending portion is selectively bendable in the upward and downward directions.

The lever is configured to be selectively tiltable in a third direction corresponding to a leftward direction in the bending directions of the bending portion or a fourth direction corresponding to a rightward direction in the bending directions via a second rotation pivot provided at a tilting support. Consequently, the bending portion is selectively bendable in the leftward and rightward directions.

Furthermore, the lever is configured to be selectively tiltable in a fifth direction that is a composite direction of the first direction and the third direction, a sixth direction that is a composite direction of the second direction and the fourth direction, a seventh direction that is a composite direction of the first direction and the fourth direction and an eighth direction that is a composite direction of the second direction and the third direction via the first rotation pivot and the second rotation pivot. Accordingly, the bending portion is selectively bendable in a composite direction of two directions of the upward, downward, leftward and rightward directions.

Here, as stated above, the lever is tiltable in a plurality of directions. An operator generally performs an operation of tilting the lever with his/her thumb. In many cases, an operation of tilting the lever with a thumb of an operator draws a trajectory close to a circular motion.

### SUMMARY OF THE INVENTION

An endoscope according to an aspect of the present invention includes: an insertion portion including an actively bendable bending portion; a lever configured to, in order to selectively bend the bending portion in any one of upward, downward, leftward and rightward directions or a composite direction of two directions of the upward, downward, leftward and rightward directions, be selectively tiltable in any one of a first direction corresponding to the upward direction, a second direction corresponding to the downward direction, a third direction corresponding to the leftward direction and a fourth direction corresponding to the rightward direction or a composite direction of two directions of the first to fourth directions and be capable of, by being tilted, pulling a bending operation wire configured to bend the bending portion; and a bending operation mechanism including a play part configured to, when the lever is tilted in the third direction or the fourth direction or the composite direction, maintain the bending portion in a state of not bending in any of the leftward direction, the rightward direction or the composite direction, up to a predetermined tilting amount. The bending operation mechanism includes a sub-rotation pivot provided at a position away in an axial direction of the lever from tilting supports of the lever in the respective tilting directions and configured to selectively tilt the lever in the third direction or the fourth direction, and a stopper configured to, when the lever is rotated by a predetermined angle corresponding to the predetermined tilting amount via rotation of the sub-rotation pivot, stop the rotation of the sub-rotation pivot through abutment with the lever. The play part is defined according to the predetermined tilting amount of the lever until the lever abuts against the stopper.

An endoscope according to another aspect of the present invention includes: an insertion portion including an actively bendable bending portion; a joystick configured to, in order to selectively bend the bending portion in any one of upward, downward, leftward and rightward directions or a composite direction of two directions of the upward, downward, leftward and rightward directions, be selectively tiltable in any one of a first direction corresponding to the upward direction, a second direction corresponding to the downward direction, a third direction corresponding to the leftward direction and a fourth direction corresponding to the rightward direction or a composite direction of two directions of the first to fourth directions, the joystick making the bending portion bend by being tilted; and a bending operation mechanism including a play part configured to, when the joystick is tilted in the third direction or the fourth direction or the composite direction, maintain the bending portion in a state of not bending in any of the leftward direction, the rightward direction or the composite direction, up to a predetermined tilting amount.

### BRIEF DESCRIPTION OF THE DRAWINGS

FIG. 1 is a plan view illustrating an endoscope of a first embodiment;

FIG. 2 is a side view of an operation portion body of an operation portion and a part of a universal cord in the endoscope in FIG. 1 in the II direction in FIG. 1;

FIG. 3 is an enlarged perspective view illustrating a bending operation device provided inside the operation portion in FIG. 1;

FIG. 4 is a perspective view of the bending operation device in FIG. 3 with a housing, a stay and a suction cylinder excluded, in the IV direction in FIG. 3;

FIG. 5 is an exploded perspective view of the bending operation device illustrated with the stay, the suction cylinder and bending operation wires removed from the bending operation device in FIG. 3;

FIG. 6 is a diagram schematically illustrating a cross-section of the bending operation device along line VI-VI in FIG. 3;

FIG. 7 is a schematic side view of the housing, a rotation frame and a lever in the bending operation device in the VII direction in FIG. 6;

FIG. 8 is a top view of only stoppers of the rotation frame and the lever in FIG. 7 in the VIII direction in FIG. 7;

FIG. 9 is a side view illustrating a shape of a modification of the stoppers in FIG. 8;

FIG. 10 is a side view illustrating a shape of a modification of the stoppers in FIG. 8, the modification being different from the modification in FIG. 9;

FIG. 11 is a diagram schematically illustrating an angle of abutment of the lever with a stopper in FIG. 9;

FIG. 12 is a top view illustrating a shape of a modification of the stoppers, the modification providing effects that are similar to effects of the configuration in FIG. 9;

FIG. 13 is a top view illustrating a shape of a modification of the stoppers, the modification providing effects that are similar to effects of the configuration in FIG. 11; and

FIG. 14 is a diagram schematically illustrating a cross-section of a bending operation device of an endoscope of a second embodiment.

### DETAILED DESCRIPTION OF THE PREFERRED EMBODIMENTS

Embodiments of the present invention will be described below with reference to the drawings.

### First Embodiment

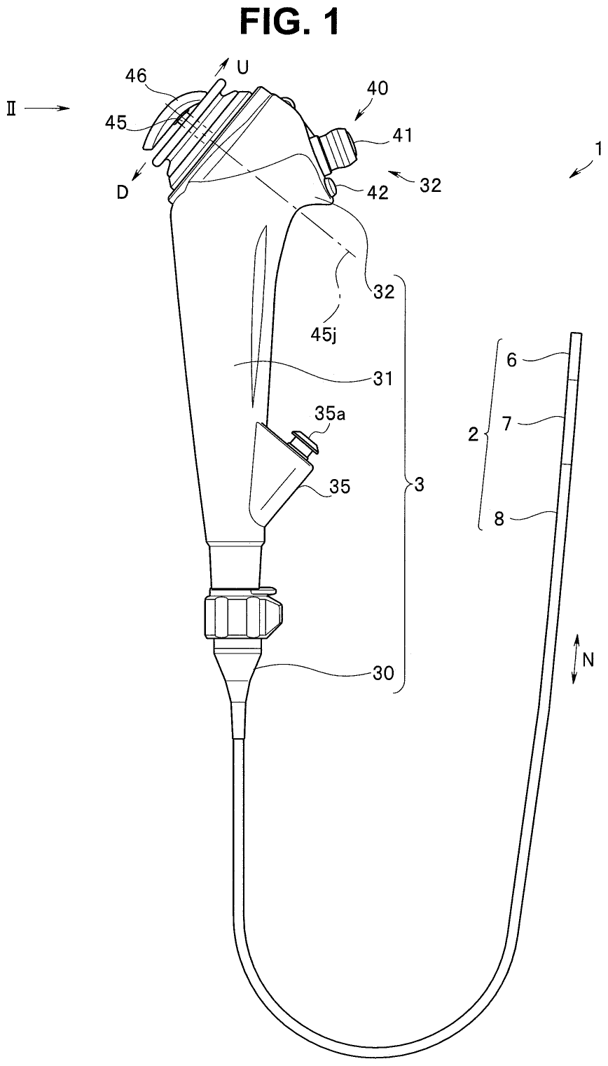

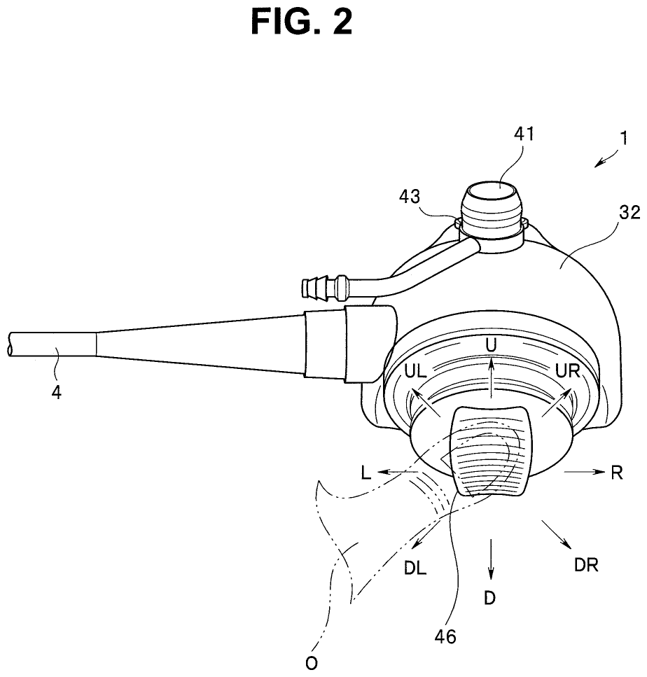

FIG. 1 is a plan view illustrating an endoscope of the present embodiment and FIG. 2 is a side view of an operation portion body of an operation portion and a part of a universal cord in the endoscope in FIG. 1 in the II direction in FIG. 1.

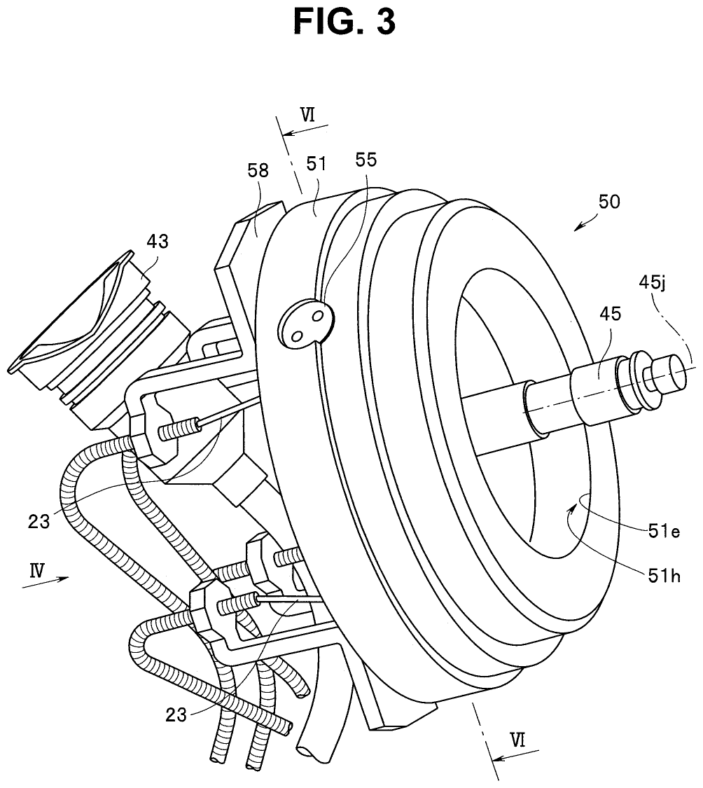

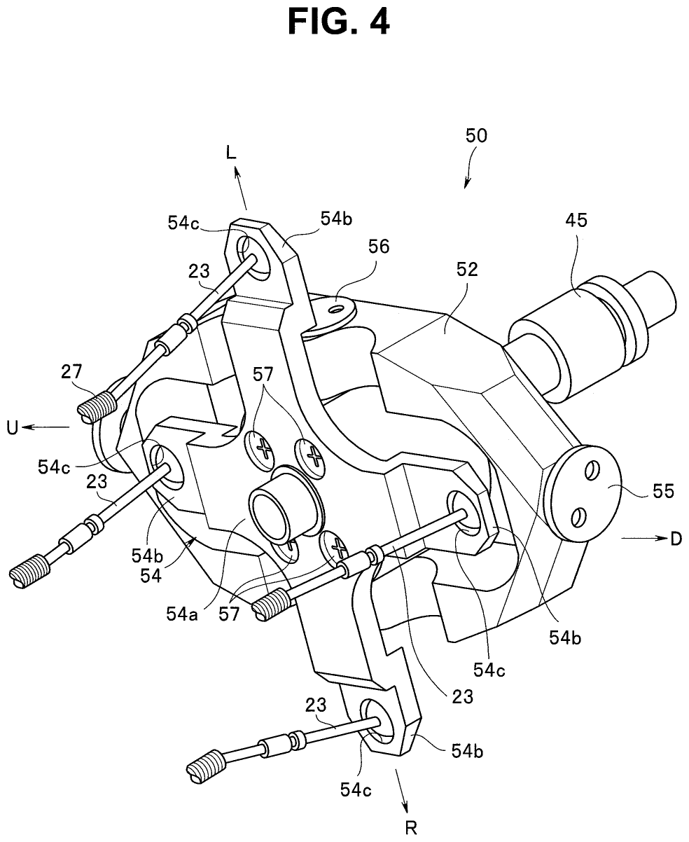

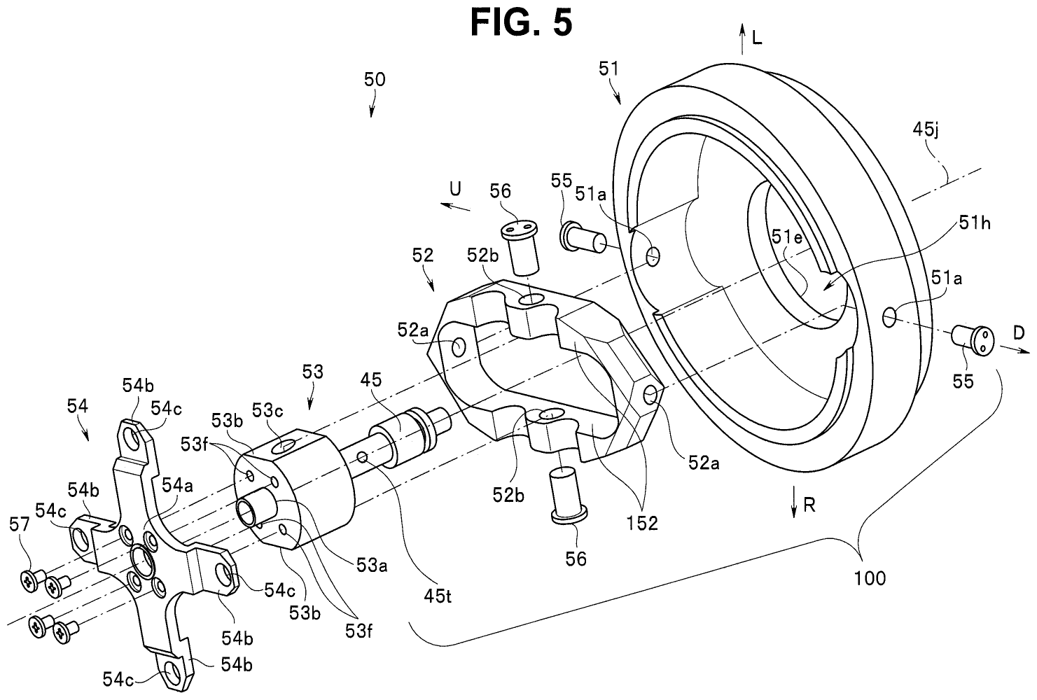

FIG. 3 is an enlarged perspective view illustrating a bending operation device provided inside the operation portion in FIG. 1, FIG. 4 is a perspective view of the bending operation device in FIG. 3 with a housing, a stay and a suction cylinder removed, in the IV direction in FIG. 3, and FIG. 5 is an exploded perspective view of the bending operation device illustrated with the stay, the suction cylinder and bending operation wires removed from the bending operation device in FIG. 3.

As illustrated in FIG. 1, an endoscope 1 includes an insertion portion 2 elongated in a longitudinal axis direction N, and an operation portion 3 continuously provided on the proximal end side in the longitudinal axis direction N of the insertion portion 2. The endoscope 1 is configured by, for example, a known ureteroscope.

The insertion portion 2 is a tubular member having flexibility and is configured by a distal end portion 6, an actively bendable bending portion 7 and a flexible tube portion 8 being continuously provided in the order mentioned from the distal end side in the longitudinal axis direction N.

Inside the distal end portion 6, e.g., an image pickup unit configured to observe, or pick up an image of, the inside of a subject and an illumination unit configured to provide illuminating light to the inside of the subject (neither of which is illustrated) are provided.

In a distal end surface of the distal end portion 6, a distal end opening (not illustrated) of a treatment instrument insertion channel is formed. The treatment instrument insertion channel is provided at least inside the insertion portion 2 and the operation portion 3 and is used for inserting and removing a treatment instrument to/from the inside of the subject and sucking a fluid inside the subject.

The bending portion 7 includes inside thereof a plurality of bending pieces joined in the longitudinal axis direction N inside. In the bending portion 7, later-described four bending operation wires (hereinafter simply referred to as “wires”) 23 (see FIG. 3) are each connected to a bending piece located on the most distal end side in the longitudinal axis direction N of the plurality of bending pieces in such a manner that respective distal ends of the wires in the longitudinal axis direction N are shifted from one another by 90° in a circumferential direction of the bending piece.

Therefore, by any one of the four wires 23 or any two of the four wires 23 being selectively pulled by a later-described bending operation device 50 (see FIG. 3), the bending portion 7 selectively or actively bends in any one of upward, downward, leftward and rightward directions or a composite direction of two directions of the upward, downward, leftward and rightward directions.

In the present embodiment, the upward, downward, leftward and rightward directions in which the bending portion 7 bends are directions defined in association with upward, downward, leftward and rightward directions in an observation image picked up by the image pickup unit provided inside the distal end portion 6.

The flexible tube portion 8 is configured by a flexible tubular member that is passively bendable. Inside the flexible tube portion 8, e.g., the aforementioned respective wires 23, and a signal cable extending out from the image pickup unit, a light guide configured to supply illuminating light to the illumination unit, and the treatment instrument insertion channel (none of which is illustrated) are inserted.

A main portion of the operation portion 3 includes a bend preventing portion 30, a grasping portion 31 and an operation portion body 32.

The bend preventing portion 30 is connected to the flexible tube portion 8 in such a manner as to cover a proximal end in the longitudinal axis direction N of the flexible tube portion 8.

The grasping portion 31 is a part to be grasped by a hand of an operator and is provided in such a manner as to be continuous with a proximal end in the longitudinal axis direction N of the bend preventing portion 30.

The grasping portion 31 has a shape that enables the operator to grasp and operate the grasping portion 31 with either the left hand or the right hand.

Furthermore, a treatment instrument insertion portion 35 is provided on the distal end side in the longitudinal axis direction N of the grasping portion 31. The treatment instrument insertion portion 35 includes a treatment instrument insertion portion opening 35a that communicates with the aforementioned treatment instrument insertion channel, and any one of various treatment instruments is inserted and removed to/from the treatment instrument insertion channel via the treatment instrument insertion portion opening 35a.

Note that a non-illustrated forceps plug for closing the treatment instrument insertion portion opening 35a is detachably attached to the treatment instrument insertion portion opening 35a.

The operation portion body 32 is provided in such a manner as to be continuous with a proximal end in the longitudinal axis direction N of the grasping portion 31. Note that the universal cord 4 (see FIG. 2) extends out from the operation portion body 32 and a non-illustrated endoscope connector is provided at an extending end of the universal cord 4.

An operation button group 40 for performing various operations of the endoscope 1 is provided on one side-surface side of the operation portion body 32.

A main portion of the operation button group 40 is configured by a suction button 41 and a button switch 42.

The suction button 41 is detachably attached to a suction cylinder 43 (see FIG. 3) provided in the operation portion body 32. As illustrated in FIG. 3, the cylinder 43 is provided inside the operation portion body 32.

The button switch 42 is configured by, for example, two button switches 42 and any function of various functions of the endoscope 1 is assigned to each button switch 42.

A lever 45 including a finger rest portion 46 in the bending operation device 50 for performing an operation of actively bending the bending portion 7 is provided on the other side-surface side of the operation portion body 32. A part around an extension part of the lever 45, the extension part being exposed from the operation portion body 32, may be covered by a known boot cover in such a manner that the finger rest portion 46 is exposed.

The bending operation device 50 is provided in the operation portion body 32. As illustrated in FIGS. 3 to 5, a main portion of the bending operation device 50 is configured by the lever 45, a bending operation mechanism 100, a swinging member 53, a wire pulling member 54, a stay 58 and the four wires 23. Note that the bending operation device 50 is configured by a known joystick device.

The lever 45 is a joystick-type operation lever configured to, in order to selectively bend the bending portion 7 in any one of the upward, downward, leftward and rightward directions or a composite direction of two directions of the upward, downward, leftward and rightward directions, be selectively tiltable in any one of a first direction U corresponding to the upward direction of the bending portion 7, a second direction D corresponding to the downward direction of the bending portion 7, a third direction L corresponding to the leftward direction of the bending portion 7 and a fourth direction R corresponding to the rightward direction of the bending portion 7 or a composite direction of two directions of the first to fourth directions U to R, from an upstanding state in an axial direction 45j, and be capable of, by being tilted, pulling any one of the wires 23.

Therefore, when the lever 45 is tilted in the first direction U in FIG. 2, the bending portion 7 bends in the upward direction, when the lever 45 is tilted in the second direction D in FIG. 2, the bending portion 7 bends in the downward direction, when the lever 45 is tilted in the third direction L in FIG. 2, the bending portion 7 bends in the leftward direction, and when the lever 45 is tilted in the fourth direction R in FIG. 2, the bending portion 7 bends in the rightward direction.

When the lever 45 is tilted in a fifth direction UL that is a composite direction of the first direction U and the third direction L, the bending portion 7 bends in a composite direction of the upward direction and the leftward direction, and when the lever 45 is tilted in a sixth direction DR that is a composite direction of the second direction D and the fourth direction R, the bending portion 7 bends in a composite direction of the downward direction and the rightward direction.

When the lever 45 is tilted in a seventh direction UR that is a composite direction of the first direction U and the fourth direction R, the bending portion 7 bends in a composite direction of the upward direction and the rightward direction, and when the lever 45 is tilted in an eighth direction DL that is a composite direction of the second direction D and the third direction L, the bending portion 7 bends in a composite direction of the downward direction and the leftward direction.

The finger rest portion 46 is a part on which, e.g., a thumb O (see FIG. 2) of an operator grasping the grasping portion 31 is placed, and is provided at a projecting end of the lever 45, the projecting end projecting to the outside of the operation portion body 32 in the axial direction 45j.

The bending operation mechanism 100 includes a housing 51 and a rotation frame 52.

As illustrated in FIGS. 3 and 5, the housing 51 is formed in a substantially cylindrical shape. An opening portion 51h is formed at a center portion of the housing 51.

The opening portion 51h allows the lever 45 to extend through the housing 51 in the axial direction 45j and an outer-side ridge 51e of the opening portion 51h configures a part that the lever 45 abuts against when the lever 45 is tilted.

In other words, by the lever 45 abutting against the outer-side ridge 51e of the opening portion 51h, the lever 45 reaches a maximum tilting angle.

As illustrated in FIG. 5, pivot holes 51a facing each other are provided in an outer peripheral portion of the housing 51.

Pins 55 configuring a second rotation pivot that serves as a tilting support when the lever 45 is selectively tilted in the third direction L or the fourth direction R to selectively bend the bending portion 7 in the leftward direction or the rightward direction are fitted in the respective pivot holes 51a. Each pin 55 is disposed in parallel to the first direction U and the second direction D.

As illustrated in FIG. 5, the rotation frame 52 is a frame body that formed in, for example, a substantially rectangular shape and includes a through hole having a predetermined shape.

A pair of locking holes 52a facing each other are provided in an outer peripheral portion of the rotation frame 52. In the outer peripheral portion of the rotation frame 52, a pair of pivot holes 52b facing each other are provided at respective positions shifted by 90° in an outer circumferential direction from respective positions of the pair of locking holes 52a.

Pins 56 configuring a first rotation pivot that serves as a tilting support when the lever 45 is selectively tilted in the first direction U or the second direction D to selectively bend the bending portion 7 in the upward direction or the downward direction are fitted in the respective pivot holes 52b. Each pin 56 is disposed in parallel to the third direction L and the fourth direction R.

The pins 55 inserted through the respective pivot holes 51a of the housing 51 are fitted in the respective locking holes 52a. As a result, the rotation frame 52 is supported in such a manner as to be rotatable in the third direction L and the fourth direction R relative to the housing 51.

As illustrated in FIG. 5, the swinging member 53 is formed in a substantially columnar shape, and in a center portion of the swinging member 53, a fitting hole 53a is formed in such a manner as to extend through the swinging member 53 in the axial direction 45j.

The proximal end side of the lever 45 is fitted in the fitting hole 53a and the swinging member 53 is integrally joined to the lever 45 by, e.g., bonding or screw-fastening.

A pair of flat portions 53b facing each other is formed at a circumferential portion of the swinging member 53. Engagement holes 53c facing each other (one engagement hole 53c is illustrated alone in FIG. 5) are provided in the respective flat portions 53b. Furthermore, for example, four screw holes 53f are formed in an end surface in the axial direction 45j of the swinging member 53.

The pins 56 inserted through the respective pivot holes 52b of the rotation frame 52 are fitted in the respective engagement holes 53c. Aa a result, the swinging member 53 and the lever 45 are supported in such a manner as to be rotatable in the first direction U and the second direction D relative to the rotation frame 52.

As described above, by the swinging member 53 being supported by the housing 51 via the rotation frame 52, the lever 45 joined to the swinging member 53 is tiltable in any one of the first to eighth directions U to DL via the pins 55, 56 inside the opening portion 51h.

As illustrated in FIG. 5, the wire pulling member 54 is formed of a plate-like member and includes arm portions 54b extending in four directions that are different from one another.

In the present embodiment, an angle formed by the respective mutually adjacent arm portions 54b provided in the wire pulling member 54 is set at 90 degrees. Therefore, the wire pulling member 54 is formed of a plate-like member having a cruciform shape in plan view.

A center portion 54a of the wire pulling member 54 is fitted to the swinging member 53 by screws 57 being threadably connected to the screw holes 53f. As a result, the lever 45 is joined to the wire pulling member 54 via the swinging member 53.

As illustrated in FIG. 4, wire attachment holes 54c are provided on the end portion sides of the respective arm portions 54b. The wires 23 are fixed to the respective wire attachment holes 54c.

Therefore, when an operator performs an operation of tilting the lever 45 in a desired direction, the wire pulling member 54 swings along with the tilting operation. Then, a wire 23 corresponding to the tilting direction of the lever 45 is pulled by the swing of the wire pulling members 54 and the bending portion 7 bends in the tilting direction of the lever 45.

More specifically, when the lever 45 is tilted in the first direction U or the second direction D by the operator until the lever 45 abuts against the outer-side ridge 51e, the wire pulling member 54 is swung in the first direction U or the second direction D by the pins 56 relative to the rotation frame 52 via the swinging member 53.

As a result, a wire 23 corresponding to the first direction U or the second direction D is pulled and the bending portion 7 bends in the upward direction or the downward direction.

When the lever 45 is tilted in the third direction L or the fourth direction R by the operator until the lever 45 abuts against the outer-side ridge 51e, the rotation frame 52 is swung in the third direction L or the fourth direction R by the pins 55 relative to the housing 51 and the wire pulling member 54 is swung in the third direction L or the fourth direction R via the swinging member 53.

As a result, a wire 23 corresponding to the third direction L or the fourth direction R is pulled and the bending portion 7 bends in the leftward direction or the rightward direction.

Furthermore, when the lever 45 is tilted in the fifth direction UL or the sixth direction DR by the operator until the lever 45 abuts against the outer-side ridge 51e, the wire pulling member 54 is swung in the fifth direction UL or the sixth direction DR by the pins 55, 56 via the swinging member 53.

As a result, two wires 23 corresponding to the fifth direction UL or the sixth direction DR are pulled simultaneously and the bending portion 7 bends in a composite direction of the upward direction and the leftward direction or a composite direction of the downward direction and the rightward direction.

When the lever 45 is tilted in the seventh direction UR or the eighth direction DL by the operator until the lever 45 abuts against the outer-side ridge 51e, the wire pulling member 54 is swung in the seventh direction UR or the eighth direction DL by the pins 55, 56 via the swinging member 53.

As a result, two wires 23 corresponding to the seventh direction UR or the eighth direction DL are pulled simultaneously and the bending portion 7 bends in a composite direction of the upward direction and the rightward direction or a composite direction of the downward direction and the leftward direction.

As illustrated in FIG. 3, the bending operation device 50 is disposed in such a manner as to face the cylinder 43 inside the operation portion body 32. More specifically, the cylinder 43 is disposed between two arm portions 54b of the wire pulling member 54.

As illustrated in FIG. 3, the stay 58 extending to the cylinder 43 side is provided at the housing 51. Guide coils covering outer circumferences of the respective wires 23 are fixed to the stay 58.

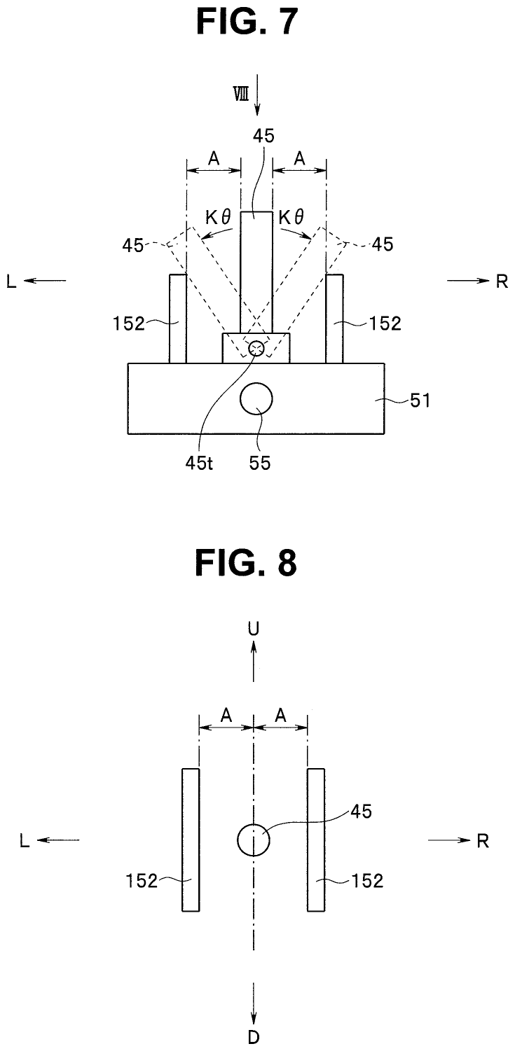

Here, the bending operation mechanism 100 includes a play part A (see FIG. 7) configured to, when the lever 45 is tilted in any one of the third to eighth directions L to DL, maintain the bending portion 7 in a state of not bending in any of the leftward direction, the rightward direction or the composite direction, up to a predetermined tilting amount.

The play part A indicates a range in which the lever 45 tilts in any one of the third to eighth directions L to DL before the bending portion 7 bending in conjunction with swing of the wire pulling member 54 when the lever 45 is tilted in any one of the third to eighth directions L to DL.

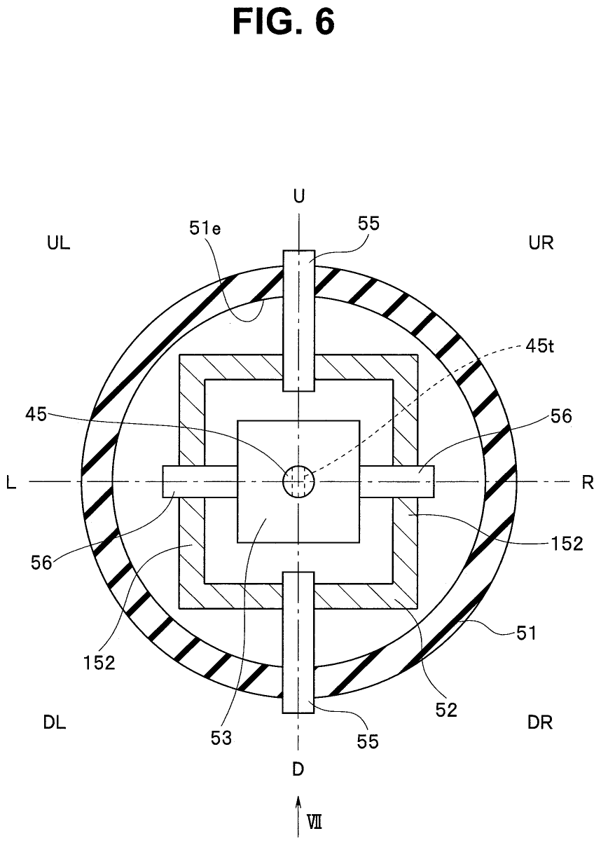

A configuration of the bending operation mechanism 100, the configuration including the play part A, will be described below with reference to FIGS. 6 to 8 in addition to FIGS. 2 and 5.

FIG. 6 is a diagram schematically illustrating a cross-section of the bending operation device along line VI-VI in FIG. 3, FIG. 7 is a schematic side view of the housing, the rotation frame and the lever in the bending operation device in FIG. 6 in the VII direction in FIG. 6, and FIG. 8 is a top view of only stoppers of the rotation frame and the lever in FIG. 7 in the VIII direction in FIG. 7.

As illustrated in FIG. 5, the bending operation mechanism 100 in the bending operation device 50 includes a third rotation pivot 45t, which is a sub-rotation pivot configured to selectively tilt the lever 45 in the third direction L or the fourth direction R, at a position away in the axial direction 45j from the pins 55, 56, which are tilting supports of the lever 45, in the lever 45.

Like the pins 55, the third rotation pivot 45t is disposed in parallel to the first direction U and the second direction D.

A rotation torque of the third rotation pivot 45t is set to be smaller than a rotation torque of the pins 55. Note that setting of each rotation torque can be made according to, for example, sliding friction at the time of rotation.

Accordingly, rotation of the rotation frame 52 in the third direction L and the fourth direction R relative to the housing 51 using the pins 55 is more difficult than rotation of the lever 45 in the third direction L and the fourth direction R using the third rotation pivot 45t.

In other words, when the lever 45 is tilted in any one of the third to eighth directions L to DL by the operator using the finger rest portion 46, rotation of the third rotation pivot 45t starts earlier than rotation of the pins 55.

Furthermore, as illustrated in FIGS. 5 to 8, in the bending operation mechanism 100, a pair of stoppers 152 configured to, when the lever 45 has been rotated by a predetermined angle Kθ corresponding to the predetermined tilting amount by rotation of the third rotation pivot 45t, stop the rotation of the third rotation pivot 45t through abutment with the lever 45, is configured as parts of the rotation frame 52.

The stoppers 152 are provided in a linear fashion at respective positions at which the stoppers 152 are parallel to the third rotation pivot 45t in the rotation frame 52. The stoppers 152 may be parts of the rotation frame 52, the parts being parallel to the third rotation pivot 45t. The stoppers 152 may be provided separately from the rotation frame 52.

The predetermined angle Kθ can be set to, for example, any angle in a range of 1° to 20°. Furthermore, the predetermined angle Kθ can be set to any angle according to a height of projection of the lever 45 from the stoppers 152 in the axial direction 45j.

Therefore, as illustrated in FIGS. 7 and 8, the play part A is configured according to the predetermined tilting angle (predetermined angle Kθ) of the lever 45 until the lever 45 abuts against a stopper 152.

As above, even if the lever 45 is tilted in any one of the third to eighth directions L to DL, the lever 45 is merely tilted by the third rotation pivot 45t until the lever 45 abuts against a stopper 152, and thus, the bending portion 7 does not bend in any of the leftward direction, the rightward direction or the composite direction.

When the lever 45 is tilted in the fifth to eight directions UL to DL, the bending portion 7 starts bending in the upward direction because of rotation of the pins 56 if the lever 45 is tilted in the fifth direction UL or the seventh direction UR, and the bending portion 7 starts bending in the downward direction because of rotation of the pins 56 if the lever 45 is tilted in the sixth direction DR or the eighth direction DL, until the lever 45 abuts against a stopper 152.

Subsequently, after the lever 45 abutting against the stopper 152, if the lever 45 is tilted in any one of the third to eighth directions L to DL, with the abutment maintained, until the lever 45 abuts against the outer-side ridge 51e, the wire pulling member 54 is swung and the bending portion 7 is bent in any of the leftward direction, the rightward direction or the composite direction.

Note that the rest of configuration of the bending operation mechanism 100 is the same as the conventional configuration.

As described above, in the present embodiment, the bending operation mechanism 100 includes the play part A that maintains the bending portion 7 in a state of not bending in any of the leftward direction, the rightward direction or the composite direction, up to the predetermined angle Kθ, when the lever 45 is tilted in any one of the third to eighth directions L to DL.

The play part A is defined according to the predetermined tilting amount (predetermined angle Kθ) of the lever 45 until the lever 45 abuts against a stopper 152.

Accordingly, when the lever 45 is intended to be tilted in either one of the first and second directions U and D, which are frequently used, even if the lever 45 is unintentionally tilted in the third to eighth directions L to DL, until the lever 45 abuts against a stopper 152, the bending portion 7 does not bend in a direction other than the upward and downward directions because of the play part A in the third direction L and the fourth direction R.

Even if the lever 45 is unintentionally tilted in either one of the fifth and seventh directions UL and UR, which are close to the first direction U, until the lever 45 abuts against the relevant stopper 152, the bending portion 7 bends in the upward direction, and even if the lever 45 is unintentionally tilted in either one of the sixth and direction DR and the eighth direction DL, which are close to the second direction D, until the lever 45 abuts against the relevant stopper 152, the bending portion 7 bends in the downward direction, and thus, the bending portion 7 can be easily bent in either one of the upward and downward directions.

Therefore, there is no need to make fine adjustment of a bending direction in either one of the upward and downward directions again as a result of the bending portion 7 being bent in a direction that is different from the upward and downward directions, preventing an increase in time of various observations and examinations.

As above, it is possible to provide the endoscope 1 including a configuration capable of, when the lever 45 configured to bend the bending portion 7 is tilted in the first direction U or the second direction D, allowing an unintentional operation of tilting the lever 45 in the third direction L, the fourth direction R or the composite direction.

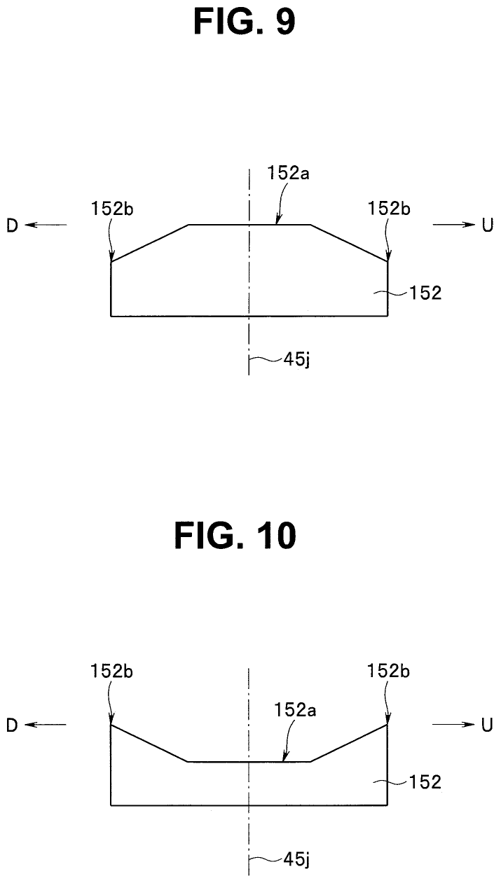

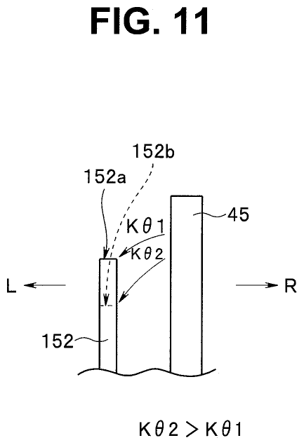

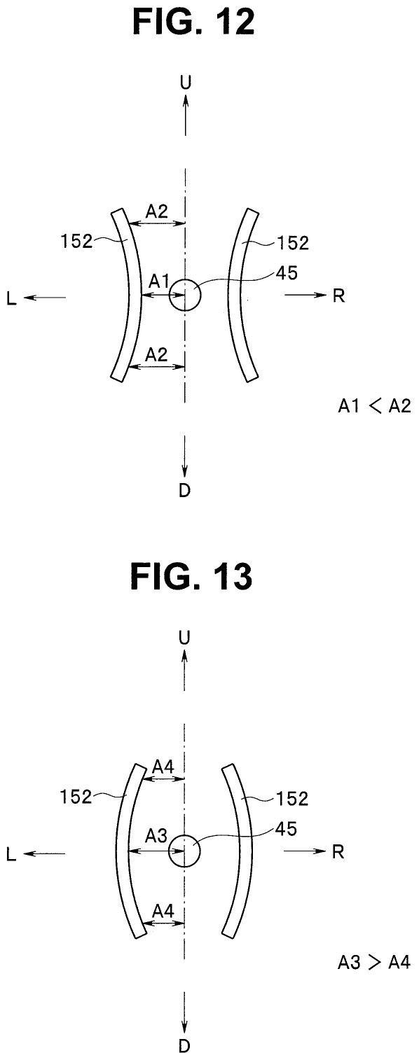

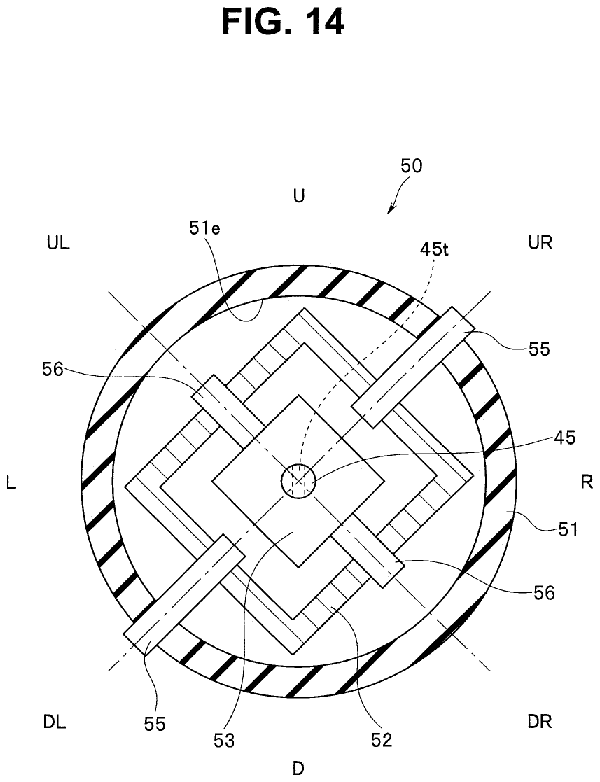

Modifications will be described with reference to FIGS. 9 to 13. FIG. 9 is a side view illustrating a shape of a modification of the stoppers in FIG. 8, FIG. 10 is a side view illustrating a shape of a modification of the stoppers in FIG. 8, the modification being different from the modification in FIG. 9, FIG. 11 is a diagram schematically illustrating an angle of abutment of a lever with a stopper in FIG. 9, FIG. 12 is a top view illustrating a shape of a modification of stoppers, the modification providing effects that are similar to effects of the configuration in FIG. 9, and FIG. 13 is a top view illustrating a shape of a modification of the stoppers, the modification providing effects that are similar to effects of the configuration in FIG. 11.

As described above, in the present embodiment, the stoppers 152 are provided in a linear fashion in the rotation frame 52 at the positions at which the stoppers 152 are parallel to the third rotation pivot 45t.

The present invention is not limited to this example and the stoppers 152 may be formed in a shape in which a predetermined angle Kθ until the lever 45 abuts against the stopper 152 varies as the stoppers 152 are further away in the first direction U or the second direction D from the lever 45.

More specifically, for example, when the lever 45 is tilted in the third direction L or the fourth direction R after the lever 45 being tilted in the first direction U or the second direction D, if the lever 45 is frequently tilted in the third direction L or the fourth direction R in a state in which an angle of the tilting in the first direction U or the second direction D is small, as illustrated in FIG. 9, each of stoppers 152 may have a shape in which a height in an axial direction 45j of the stopper 152 decreases toward respective end portions 152b as the stopper 152 is further away from an upstanding position 152a of the lever 45 in the first direction U or the second direction D.

In this case, as illustrated in FIG. 11, a tilting angle Kθ2 of a lever 45 on each end portion 152b side until the lever 45 abuts against the relevant stopper 152 is larger than a tilting angle Kθ1 of the lever 45 at a position 152a (Kθ2>Kθ1), and thus, the lever 45 less easily hits the relevant stopper 152 further on the end portion 152b side. In other words, a play part A becomes larger further on the end portion 152b side.

Since movement of a finger more largely deviates in the third direction L or the fourth direction R further on the end portion 152b side than at the position 152a, the above configuration enables such deviation to be absorbed by the large play part A.

On the other hand, for example, when the lever 45 is tilted in the third direction L or the fourth direction R after the lever 45 being tilted in the first direction U or the second direction D, if the lever 45 is frequently tilted in the third direction L or the fourth direction R in a state in which an angle of the tilting in the first direction U or the second direction D is large, as illustrated in FIG. 10, each of stoppers 152 may have a shape in which a height in an axial direction 45j of the stopper 152 increases toward respective end portions 152b as the stopper 152 is further away from an upstanding position 152a of the lever 45 in the first direction U or the second direction D.

In this case, unlike in FIGS. 9 and 10, an angle of tilting the lever 45 on each end portion 152b side until the lever 45 abuts against the relevant stopper 152 is smaller than a tilting angle of the lever 45 at the position 152a, and thus, the lever 45 more easily hits the relevant stopper 152 further on the end portion 152b side. In other words, the play part A is smaller further on the end portion 152b side.

As illustrated in FIG. 12, stoppers 152 can provide effects that are similar to the effects of the shape of the stopper 152 in FIG. 9 if each of the stoppers 152 has a planar-view shape in which a distance until a lever 45 abuts against the stopper 152 (an amount of the play part A) increases as away from the neutral position of the lever 45 in the first direction U or the second direction D (A2>A1).

Furthermore, as illustrated in FIG. 13, stoppers 152 can provide effects that are similar to the effects of the shape of the stopper 152 in FIG. 10 if each of the stoppers 152 has a planar-view shape in which a distance until a lever 45 abuts against the stopper 152 (the amount of the play part A) increases as close to the neutral position of the lever 45 in the first direction U and the second direction D (A3>A4).

### Second Embodiment

FIG. 14 is a diagram schematically illustrating a cross-section of a bending operation device in an endoscope according to the present embodiment.

A configuration of the endoscope of the second embodiment is different in prescribed directions of a first rotation pivot and a second rotation pivot from the configuration of the endoscope of the first embodiment described above with reference to FIGS. 1 to 8.

Therefore, only this difference will be described and components that are similar to components of the first embodiment are provided with reference numerals that are the same as the reference numerals of the components of the first embodiment and description of such components is omitted.

As illustrated in FIG. 14, in the present embodiment, in a bending operation device 50, pins 56 are disposed in parallel to a fifth direction UL and a sixth direction DR and pins 55 are disposed in parallel to a seventh direction UR and an eighth direction DL.

In other words, a lever 45 is tiltable in the seventh direction UR or the eighth direction DL relative to a rotation frame 52 via the pins 56, and furthermore, the rotation frame 52 is tiltable in the fifth direction UL and the sixth direction DR relative to a housing 51 via the pins 55 after the lever 45 being tilted.

Furthermore, the lever 45 is selectively tiltable in any one of first to eighth directions U to DL via rotation of the pins 55 and rotation of the pins 56.

More specifically, if the lever 45 is tilted in any one of the first to fourth directions U to R, both the pins 55 and the pins 56 rotate, if the lever 45 is tilted in the fifth direction UL or the sixth direction DR, the pins 55 rotate, and if the lever 45 is tilted in the seventh direction UR or the eighth direction DL, the pins 56 rotate.

Note that as in the first embodiment, a third rotation pivot 45t is disposed in parallel to the first direction U and the second direction D.

In other words, in the present embodiment as well, the lever 45 is tiltable in any one of the third to eighth directions L to DL via the third rotation pivot 45t until the lever 45 abuts against a relevant stopper 152 in such a manner that a play part A configured to maintain the bending portion 7 in a non-bending state is configured.

In the present embodiment, a rotation torque of the third rotation pivot 45t is set to be smaller than each of rotation torques of the pins 55 and the pins 56.

The bending operation device 50 having such configuration as above is provided inside an operation portion 3 where the endoscope 1 is configured by, for example, a bronchoscope.

Note that the rest of configuration is the same as the rest of configuration of the first embodiment described above.

In such configurations as above, the bending operation mechanism 100 can provide effects that are similar to the effects of the first embodiment described above because of including the play part A configured to, when the lever 45 is tilted in any one of the third to eighth directions L to DL, maintain the bending portion 7 in a state of not bending in any of a leftward direction, a rightward direction or a composite direction, up to a predetermined angle Kθ.

Effects that are similar to the effects of the first embodiment, such as the bending portion 7 being able to be easily bent in either one of upward and downward directions can be provided because even if the lever 45 is unintentionally tilted in either one of the fifth and seventh directions UL and UR, which are close to the first direction U, the bending portion 7 bends in the upward direction until the lever 45 abuts against a relevant stopper 152, and furthermore, even if the lever 45 is unintentionally tilted in either one of the sixth and eighth directions DR and DL, which are close to the second direction D, the bending portion 7 bends in the downward direction until the lever 45 abuts against a relevant stopper 152.

Other effects are the same as the effects of the first embodiment described above.

What is claimed is:

1. An endoscope comprising:
an insertion portion including an actively bendable bending portion;
a lever configured to, in order to selectively bend the bending portion in any one of upward, downward, leftward and rightward directions or a composite direction of two directions of the upward, downward, leftward and rightward directions, be selectively tiltable in any one of a first direction corresponding to the upward direction, a second direction corresponding to the downward direction, a third direction corresponding to the leftward direction and a fourth direction corresponding to the rightward direction or a composite direction of two directions of the first to fourth directions and be capable of, by being tilted, pulling a bending operation wire configured to bend the bending portion; and
a bending operation mechanism including a play part configured to, when the lever is tilted in the third direction or the fourth direction or the composite direction, maintain the bending portion in a state of not bending in any of the leftward direction, the rightward direction or the composite direction, up to a predetermined tilting amount, wherein:
the bending operation mechanism includes
a sub-rotation pivot provided at a position away in an axial direction of the lever from tilting supports of the lever in the respective tilting directions and configured to selectively tilt the lever in the third direction or the fourth direction, and
a stopper configured to, when the lever is rotated by a predetermined angle corresponding to the predetermined tilting amount via rotation of the sub-rotation pivot, stop the rotation of the sub-rotation pivot through abutment with the lever, and
the play part is defined according to the predetermined tilting amount of the lever until the lever abuts against the stopper.

2. The endoscope according to claim 1,
wherein the bending operation mechanism includes, as the tilting supports,
a first rotation pivot for causing the lever to selectively tilt in the first direction or the second direction and causing the bending portion to selectively bend in the upward direction or the downward direction; and
a second rotation pivot for causing the lever to selectively tilt in the third direction or the fourth direction and causing the bending portion to selectively bend in the leftward direction or the rightward direction.

3. The endoscope according to claim 2, wherein:
the first rotation pivot is disposed in parallel to the third direction and the fourth direction; and
the second rotation pivot and the sub-rotation pivot are disposed in parallel to the first direction and the second direction.

4. The endoscope according to claim 3, wherein a rotation torque of the sub-rotation pivot is smaller than a rotation torque of the second rotation pivot.

5. The endoscope according to claim 1, wherein:
the bending operation mechanism includes, as the tilting supports,
a first rotation pivot disposed in parallel to a fifth direction that is a composite direction of the first direction and the third direction and a sixth direction that is a composite direction of the second direction and the fourth direction, and
a second rotation pivot disposed in parallel to a seventh direction that is a composite direction of the first direction and the fourth direction and an eighth direction that is a composite direction of the second direction and the third direction; and
the lever is selectively tiltable in any one of the first to eighth directions via rotation of the first rotation pivot and rotation of the second rotation pivot.

6. The endoscope according to claim 5, wherein the rotation torque of the sub-rotation pivot is smaller than each of rotation torques of the first rotation pivot and the second rotation pivot.

7. The endoscope according to claim 1, wherein the stopper is disposed in parallel to the sub-rotation pivot.

8. The endoscope according to claim 1, wherein the stopper is formed in a shape in which the predetermined tilting amount of the lever until the lever abuts against the stopper varies as the stopper is further away in the first direction or the second direction from the lever.

9. The endoscope according to claim 1, wherein the lever is provided on a proximal end side in a longitudinal axis direction of the insertion portion.

10. An endoscope comprising:
an insertion portion including an actively bendable bending portion;
a joystick configured to, in order to selectively bend the bending portion in any one of upward, downward, leftward and rightward directions or a composite direction of two directions of the upward, downward, leftward and rightward directions, be selectively tiltable in any one of a first direction corresponding to the upward direction, a second direction corresponding to the downward direction, a third direction corresponding to the leftward direction and a fourth direction corresponding to the rightward direction or a composite direction of two directions of the first to fourth directions, the joystick making the bending portion bend by being tilted; and
a bending operation mechanism including a play part configured to, when the joystick is tilted in the third direction or the fourth direction or the composite direction, maintain the bending portion in a state of not bending in any of the leftward direction, the rightward direction or the composite direction, up to a predetermined tilting amount.
